(19) United States
(12) Patent Application Publication
(10) Pub. No.: US 2003/0082135 A1
(43) Pub. Date: May 1, 2003

(54) Pathway of RANTES-mediated chemokine synthesis in astrocytes and methods of use therefor

(76) Inventor: Martin E. Dorf, Chestnut Hill, MA (US)

Correspondence Address: Sonia K. Guterman, Esq., MINTZ, LEVIN, COHN, FERRIS,, GLOVSKY and POPEO, P.C., One Financial Center, Boston, MA, 02111, US

(21) Appl. No.: 10/284,934

(22) Filed: Oct. 30, 2002

Related U.S. Application Data
(60) Provisional application No. 60/340,724, filed on Oct. 30, 2001.

(51) Int. Cl. A61K 38/19; C12Q 1/68; G01N 33/567

(52) U.S. Cl. 424/85.1; 435/6; 435/7.21

(57) ABSTRACT

Methods for modulating RANTES- and RANTES-related chemokine induction of expression of a family of genes in cells of the central nervous system, including genes encoding cell surface receptors, and methods of use and compositions containing such inhibitors are provided.

### RELATED APPLICATION

This application claims priority from provisional application No. 60/340,725, filed on Oct. 30, 2001, and which is incorporated in its entirety by reference herein.

### GOVERNMENT SUPPORT

This invention was made in part with government support under grants NS37284 and CA67416 from the National Institutes of Health. The government has certain rights in the invention.

### TECHNICAL FIELD

Methods of reducing inflammatory and neurodegenerative responses in parenchymal cells of the central nervous system, and methods of screening for therapeutic agents that reduce those responses are provided.

### BACKGROUND

Cells of the human central nervous system (CNS) are subject to inflammatory disorders such as demyelinating conditions, for example, multiple sclerosis, and inflammation can arise from meningitis, cerebritis, brain and spinal cord injury, stroke, and chronic neurodegenerative diseases such as Alzheimer's and Parkinson's diseases. Agents are needed that reduce inflammation in the CNS, to treat these disorders.

The chemokine RANTES (regulated on activation normal T cell expressed; also known as CCL5, and as small inducible cytokine A5 or SCYA5) is involved in the ontogenic development of the brain. A polymorphism of the gene for RANTES doubles the frequency of a severe form of asthma in humans (Fryer, A. et al. Genes Immun. 1:509, 2000). Higher levels of RANTES may be associated with more severe inflammation.

Most studies of chemokines focus on their roles in leukocyte migration and activation. However, astrocytes from 5-week-old fetal human brains release RANTES, which in turn induces proliferation of the astrocyte cultures (Bakhiet, M. 2001. Nat Cell Biol. 3:150). Further, after these cells have expanded at week 10 of gestation, RANTES synergizes with interferon-γ to inhibit the proliferative response and to promote cell survival, facilitating astrocyte differentiation. The signaling mechanisms regulating chemokine mediated effects by RANTES and related chemokines on astrocytes remain poorly defined.

Characterization of the cellular effects induced by RANTES and related chemokines can provide methods for isolation and identification of agents that might reduce inflammation of the CNS.

### SUMMARY

In one embodiment the invention features a method of reducing inflammatory responses in parenchymal cells of the central nervous system (CNS) of a subject, the method comprising providing the subject with an inhibitor of a RANTES-related chemokine binding to a high affinity chemokine receptor in the CNS, such that RANTES-related chemokine signal transduction and amplification of chemokine gene expression are inhibited, thereby reducing inflammatory responses in the cells. The chemokine is selected from the group consisting of RANTES, eotaxin, MIP-1α, and MIP-1β. In related embodiments, providing the inhibitor includes delivering it directly to the CNS, for example, by providing an additional agent that permeabilizes the blood-brain barrier, or by providing the inhibitor in a CNS implant.

The invention in another embodiment provides a method of obtaining an agent that inhibits up-regulation of expression of a proinflammatory gene in a population of activated astrocytes, comprising providing a sample of astrocytes with at least one candidate agent; testing the candidate for ability to inhibit signal transduction of the RANTES/RSK pathway, and identifying the agent as an inhibitor of a step in the pathway of the sample of astrocytes in comparison with a control sample of astrocytes not provided with the candidate and otherwise identical, such that the candidate is an inhibitor of up-regulation of a pro-inflammatory gene in astrocytes. In this method, activated astrocytes have been pretreated with a RANTES-related chemokine.

According to a related embodiment, the step of the pathway is RSK phosphorylation. Inhibiting the step of the pathway can be providing a mutant form of the RANTES-related chemokine, for example, providing a dominant negative mutant the chemokine. Alternatively, inhibiting the step of the pathway is providing an inhibitor that antagonizes binding of the chemokine to a receptor, for example, an inhibitor of RANTES binding to CCR1 or CCR5 receptor. For example, the inhibitor inhibits binding of HIV-1 to the high affinity receptor. In a related embodiment, the inhibitor is selected from the group consisting of: APO-RANTES, sCH-C, and TAK-779.

The invention in another embodiment provides a method of treating a subject having an inflammatory condition of the CNS, comprising providing according to any of the above methods an inhibitor of the RANTES/RSK signal transduction pathway; and administering to the subject a composition containing an effective dose of an inhibitor, in a pharmaceutically acceptable excipient. The inflammatory condition of the CNS is for example a demyelinating condition. The demyelinating condition is selected from the group consisting of multiple sclerosis, a post-vaccination condition, post-viral infection condition, and a post-anti TNF treatment condition. Further, in another embodiment, the inflammatory condition of the CNS is a neurodegenerative disease. The neurodegenerative disease is Alzheimer's disease or Parkinson's disease. In other embodiments, the inflammatory condition of the CNS is selected from meningitis, cerebritis, brain and spinal cord injury, and stroke. Further, the composition can comprise an additional therapeutic agent, for example, a β-interferon, or a random linear amino acid copolymer, for example, Copaxone®.

In another embodiment, the invention provides a method of screening a library of compounds to identify an inhibitor of RANTES/RSK signal transduction in a parenchymal cell of the CNS, the method comprising providing a cell with a RANTES-related chemokine and at least one of the compounds and analyzing the cell for expression of a gene that is up-regulated in response to chemokine treatment, wherein decreased expression of the gene in the presence of the compound, compared to that in a control cell similarly treated with chemokine but in the absence of the compound, indicates that the compound is an inhibitor of the pathway. The parenchymal cell is for example an astrocyte. The method may further include analyzing the cell for expression of the gene, for example, analyzing the cell is measuring an RNA transcript of the gene.

Alternatively, analyzing the cell is measuring a protein product of the gene. Alternatively, assaying for the protein product further is measuring the protein antigenically or functionally, for example, measuring the protein antigenically is performing a western blot. In another example, measuring the protein functionally is measuring a marker enzyme which is a fusion of the gene and a nucleic acid encoding the marker enzyme. Accordingly, the marker enzyme is selected from the group consisting of luciferase and β-galactosidase. Further, measuring the protein functionally is assaying for expression of a fusion of the gene with a non-enzymatic marker protein, for example, the non-enzymatic marker protein is a colored fluorescent protein. In these related methods, the gene may encode a protein which is selected from the group consisting of: TNF-α, RANTES, KC, IL-6, MIP-1α, MIP-2, MCP-1, ICAM-1, CX3CR1, and CXCR4.

In another embodiment, the invention provides a method of screening compounds to identify an inhibitor of the RANTES/RSK pathway in parenchymal cells of the CNS, the method comprising providing a RANTES-related chemokine and at least one compound to a sample of the cells and analyzing the sample of cells for phosphorylation of a protein of the pathway, wherein decreased phosphorylation of the protein in the presence of the compound, compared to that in a control sample of the cells similarly treated with RANTES but in the absence of the compound, indicates that the compound is an inhibitor of the pathway. For example, the protein is RSK, Raf-1, MEK, or protein kinase A (PKA).

In another embodiment, the invention provides a method of screening a library of compounds to identify an inhibitor of the RANTES/RSK pathway in parenchymal cells of the CNS, the method comprising providing a first cell extract from a sample of the parenchymal cells that have been pre-treated with a RANTES-related chemokine, and a second cell extract from otherwise identical control parenchymal cells which have not been pre-treated with the chemokine providing at least one candidate inhibitor compound to the first and second extracts and assaying the first and second extracts for function of a protein in the RANTES/RSK pathway in the presence and absence of the candidate inhibitor, wherein decreased function of the protein in the first cell extract in the presence of the compound, compared to that of the first cell extract in the absence of the compound and the second cell extract, indicates that the compound is an inhibitor of the pathway. Accordingly, the function of the protein is a kinase activity. Cells are exposed to chemokine at about 1 nmolar at about 2 nanomolar, or at less than about 10 nmolar. Cells are exposed to chemokine at about 100 ng/ml. Further, pretreated cells have been exposed to chemokine for at least 5 minutes.

### BRIEF DESCRIPTION OF THE DRAWINGS

FIG. 1 is a photograph of an RNA gel and two line graphs showing that RANTES induced cytokine and chemokine gene expression in astrocytes. Panel A. Astrocytes were stimulated with 100 ng/ml RANTES for the indicated times. Total RNA was prepared and assayed by RNase protection assay (RPA) for message expression of TNF-α, RANTES, KC, IL-6, MIP-1α, MIP-2, MCP-1, L32, and GAPDH. Representative data from one of three similar experiments are presented. Panel B. Kinetics of KC protein expression were determined by ELISA using culture supernatants collected at the indicated times. Panel C. Kinetics of TNF-α protein expression were determined by ELISA using culture supernatants collected at the indicated times.

FIG. 2 is a set of photographs of Western blots and a bar graph showing that RANTES induced phosphorylation of MEK and ERK1/2, but not JNK or p38. Panel A. Astrocytes were stimulated with 100 ng/ml RANTES for the indicated times and cell lysates were prepared for analysis by Western blotting. Blots were stained with anti-phospho-MEK1/2 Ab, anti-phospho-ERK1/2 Ab, and antibodies that detected total ERK1/2 expression. Panel B. Pooled data from 3 to 4 independent experiments. Gels were scanned on a phosphoimager to quantitate the data. The results were normalized based on the levels of total ERK1/2. Grey shaded bars represent phospho-MEK, solid bars are phospho-ERK1, and open bars represent phospho-ERK2± the standard error of the mean (S.E.M.). Panel C. Astrocytes were stimulated for 20 minutes with medium, 100 ng/ml RANTES, or 20 ng/ml IL-1β. Cell lysates were analyzed by Western blotting with anti-phospho-ERK1/2, anti-phospho-SAPK/JNK, and anti-phospho-p38 Ab.

FIG. 3 is a photograph of an RNA gel showing the effects of the MEK inhibitor, U0126, on induction of TNF-α and chemokine transcripts. Astrocytes were pretreated with the indicated amount of U0126 then stimulated with 100 ng/ml RANTES for 3 h and total RNA was prepared and assayed by RPA as for FIG. 1.

FIG. 4 is a bar graph showing the effect of U0126 pretreatment on RANTES induced KC reporter gene expression. Astrocytes were transfected with a luciferase reporter construct driven by the murine KC promoter. After 24 h, cells were stimulated in the absence or presence of the indicated amount of U0126 or 5 μM SB203580, an inhibitor of p38, plus 100 ng/ml RANTES for another 24 h before luciferase activity was measured. Values are presented as arbitrary luciferase units and represent the mean±S.E.M. of triplicate experiments.

FIG. 5 is a set of photographs of Western blots and bar graphs showing that RANTES induced phosphorylation of p90RSK via the MAP kinase pathway. Panel A. Astrocytes were stimulated with 100 ng/ml RANTES for the indicated time. Cell lysates were assayed by Western blotting with anti-phospho-p90RSK (Ser381), anti-phospho-p90RSK (Thr360/Ser364), and anti-phospho-p90RSK (Thr574) Ab. The same lysates were also analyzed for total expression of the kinase using anti-p90RSK antibody to ensure equal protein loading. Panel B. To quantitate the data, gels from 3 to 4 independent experiments were examined on a phosphoimager and the data were normalized based on the levels of total p90RSK protein. The shaded grey bars represent RSK phosphorylation at Ser 381, solid bars are RSK phosphorylation of Thr 360 and Ser 364, and the open bars are RSK phosphorylation at Thr 574. Panel C. Astrocytes were pretreated with the indicated concentrations of U0126, and were stimulated with 100 ng/ml RANTES for 20 min. Western blots were performed as indicated above. Panel D. Gels from at least 3 independent experiments as shown in panel C were examined on a phosphoimager and the data were normalized based on the levels of total ERK1/2 protein. Grey shaded bars represent phospho-ERK1, solid bars phospho-ERK2, and open bars phospho-RSK±S.E.M.

FIG. 6 is a bar graph that shows the effect of dominant negative p90RSK plasmid on the KC reporter gene. Astrocytes were co-transfected with the luciferase reporter construct driven by murine KC promoter and expression plasmids for the wild-type p90RSK or the kinase defective mutant of p90RSK. Transfected astrocytes were stimulated with medium (open bars) or 100 ng/ml RANTES (solid bars) for 24 h before the cells were harvested to detect luciferase activity. Values are given in arbitrary luciferase units and represent the mean±S.E.M. of triplicate experiments.

FIG. 7 is a set of microphotographs that shows RANTES mediated the translocation of phosphorylated p90RSK. Astrocytes stimulated with 100 ng/ml RANTES for 5 or 20 min were stained by anti-phospho-p90RSK (Thr360/Ser364) Ab and Hoechst 33258 (to detect nuclei).

FIG. 8 is a set of bar graphs showing that RANTES reduced intracellular cAMP accumulation in astrocytes. Panel A. Astrocytes were treated with the indicated doses of RANTES or TCA4 for 5 min. Intracellular cAMP was detected as described in Materials and Methods. Values represent the mean±S.E.M of triplicate experiments. Panel B. Kinetics of intracellular cAMP levels. Astrocytes were treated with 100 ng/ml RANTES for indicated times. Panel C. RANTES inhibited forskolin-induced intracellular cAMP accumulation. Astrocytes were pretreated with 1 μM forskolin for 1 h then stimulated with the indicated amount of RANTES for 5 min. Intracellular cAMP was determined by EIA. Values are presented as relative cAMP level and represent the mean±S.E.M. of triplicate experiments.

FIG. 9 is a photograph of an RNA gel and a bar graph showing that the effects of PTx on RANTES stimulation of astrocytes. Panel A. Astrocytes were pretreated with 1 ng/ml PTx for 1 h and then stimulated with 100 ng/ml RANTES for 3 h. Total RNA was prepared and assayed by RPA for expression of TNF-α, RANTES, KC, IL-6, MIP-1α, MIP-2, MCP-1, L32 and GAPDH message. Representative data from one of three similar experiments are presented. Panel B. Dose response of PTx on RANTES mediated modulation of intracellular cAMP. Astrocytes were precultured with the indicated doses of PTx for 1 h and then treated with or without 100 ng/ml RANTES. Intracellular cAMP was determined by EIA. Values are presented as relative cAMP level (percent) and represent the mean±S.E.M. of triplicate experiments.

FIG. 10 is a set of bar graphs and a photograph of an RNA gel showing that protein kinase A is involved in RANTES transcription in astrocytes. Panel A. RANTES decreased PKA activity in primary mouse astrocytes. Astrocytes were treated with the indicated doses of RANTES for 20 min and cell lysates were prepared for analysis of PKA activity. Values are presented as relative PKA enzyme activity (percent) and represent the mean±S.E.M. of triplicate experiments. Panel B. Astrocytes were treated with 1 μM forskolin or 500 μM db-cAMP or 500 μM 8-bromo-cAMP for 1 h and cell lysates were prepared for analysis of PKA activity. Values are presented as relative PKA enzyme activity (percent) and represent the mean±S.E.M. of triplicate experiments. Panel C. PKA inhibitors (H-89, Rp-8-bromo-cAMP or PKI) induced MIP-1α transcription. Astrocytes were treated with the indicated dose of PKA inhibitors for 3 h and total RNA was prepared and assayed by RPA as for FIG. 9. The induction of MIP-1α was normalized based on the GAPDH. Panel D. Astrocytes were treated with the indicated doses of H-89 for 3 h and total RNA was prepared and assayed by RPA. Representative data from one of three similar experiments are presented.

FIG. 11 is a set of RNA gels showing that shows cAMP inhibits transcription induced by RANTES or H-89. Panel A. Astrocytes were pretreated with 500 μM Dibutyrate cAMP or 8-bromo-cAMP for 1 h then stimulated with 100 ng/ml RANTES for 2 h. Total RNA was prepared and assayed by RPA for the indicated transcripts. Representative data from one of three similar experiments are presented. Panel B. Astrocytes were pretreated with 500 μM dibutyrate cAMP for 1 h and then stimulated with 10 μM H-89 for 3 h. Total RNA was prepared and assayed by RPA as above.

FIG. 12 is a set of bar graphs and a photograph of a Western blot showing that RANTES activated Raf-1 kinase activity in astrocytes. Panel A. Astrocytes were stimulated with 100 ng/ml RANTES for the indicated times and cell lysates were prepared for analysis of Raf-1 activity. Values are presented as relative Raf-1 kinase activity (percent) and represent the mean±S.E.M. of triplicate experiments. Panel B. Astrocytes were stimulated with 100 ng/ml RANTES for the indicated times and cell lysates were prepared for analysis by Western blotting. Blots were stained with anti phospho-Raf (Ser 259) Ab or control anti-Raf Ab. Panel C. Raf-1 inhibitor blocked RANTES-induced transcription. Astrocytes were pretreated with the indicated doses of Raf-1 inhibitor for 1 h and then stimulated with 100 ng/ml RANTES for 3 h. Total RNA was prepared and assayed by RPA for expression of message for the indicated proinflammatory mediators and the housekeeping genes L32 and GAPDH. Representative data from one of three similar experiments are presented. Panel D. Astrocytes were co-transfected with the luciferase reporter construct driven by a murine MIP-2 promoter and expression plasmids for the wild-type Raf (WT-Raf), dominant negative Raf (DN-Raf) or a constituitively active mutant of Raf (CA-Raf). Transfected astrocytes were stimulated with medium (open bar) or 100 ng/ml RANTES (shaded bar) for 8 h before the cells were harvested to detect luciferase activity. Values are given in arbitray luciferase units and represent the mean±S.E. of triplicate experiments.

FIG. 13 is a set of bar graphs, photographs of Western blots, and a photograph of an RNA gel showing that PKA inhibitors activated Raf/MAPK pathway in astrocytes. Panel A. Astrocytes were stimulated with the indicated doses PKA inhibitors for 10 min and cell lysates were prepared for analysis of Raf-1 activity. Values are presented as relative Raf-1 kinase activity (percent) and represent the mean±S.E.M. of triplicate experiments. Panel B. H-89 induced phosphorylation of MEK, erk1/2 and RSK. Astrocytes were stimulated with the indicated doses of H-89 or GF109203 for 20 min and cell lysates were prepared for analysis by Western blotting. Blots were probed with anti-phospho-MEK antibody, anti-phospho-erk1/2, anti-phospho-RSK (Ser 381) and antibodies that detected total erk1/2 expression. Panel C. Astrocytes were pretreated with the indicated concentrations of U0126 or 5 μM SB203580 and stimulated with 10 μM H-89 for 20 min. Western blots were performed as indicated above. Panel D. U0126 blocked cytokine and chemokine transcription induced by H-89. Astrocytes were pretreated for 1 h with the indicated amount of U0126 then stimulated with 10 μM H-89 for 3 h and total RNA was prepared and assayed by RPA as in FIG. 12.

### DESCRIPTION OF SPECIFIC EMBODIMENTS

Astrocytes are the most abundant cell type within the human central nervous system (CNS). These non-neuronal parenchymal cells of the CNS are capable of bidirectional communication with neurons and are thought to process information. (Cornell-Bell, et al. 1990. Science 247:470; Nedergaard, M. 1994. Science 263:1768; Parpura, V., et al. 1994. Nature 369:744.) Astrocytes also help maintain the homeostatic climate of the CNS (Norenberg, M. D. 1997. Immunology of the Nervous System 173). They express receptors for proinflammatory cytokines, bacterial products, complement components, and constituents of the coagulation system (Dorf, M. E., et al. J Neuroimmunol 111:109; Huang, D., et al. 2000. Immunol Rev 177:52; Morgan, B. P., et al. 1997. Immunopharmacology 38:43; Sayah, S., et al. 1999. J Neurochem 72:2426; Pindon, A., et al. 2000. J Neurosci 20:2543). These receptors become activated during autoimmune, infectious, inflammatory, or cerebrovascular diseases resulting in the release of cytokines and effector molecules (Huang, D., et al. 2000. Immunol Rev 177:52; Bacon, K. B., et al. 2000. J Neuroimmunol 104:92; Asensio, V. C., et al. 1999. Trends Neurosci 22:504).

Chemokines are a family of proinflammatory cytokines that stimulate directional migration of leukocytes. Chemokines are produced by a spectrum of cell types, including T-lymphocytes, macrophages, endothelial cells, microglia, and astrocytes (Janabi, N., et al. 1999. J Immunol 162:1701; Luster, A. D. 1998. N Engl J Med 338:436). Inflammatory responses in the CNS rapidly induce activation of astrocytes. Activated astrocytes are associated with the production of multiple chemokines and cytokines.

The chemokine system is also involved in other physiological and pathological processes including embryogenesis, HIV infection, and tumorigenesis (Zou, Y., et al. 1998. Nature 393:595; Ma, Q., et al. 1998. Proc Natl Acad Sci U S A 95:9448.; Feng, Y., et al. 1996. Science 272:872; Muller, A., et al. 2001. Nature 410:50).

The effects of RANTES and RANTES-related chemokines on cultured neonatal murine astrocytes as examined herein shows that the signaling pathway regulating these responses involves activation of the MEK, ERK, and RSK kinases. RANTES stimulation is shown herein to induce TNF-α and KC proteins, and to induce expression of a variety of chemokine transcripts. “RANTES-related chemokine(s)” as used herein and in the claims refers to a group of chemokines, each of which activates the RANTES/RSK pathway and induces expression of a set of genes, including genes encoding TNF-α, RANTES, KC, IL-6, MIP-1α, MIP-2 and MCP-1. RANTES-related chemokine(s) RANTES, cotaxin (also termed CCL11), MIP-1α (also known as CCL3), and MIP-1β.

The term “RANTES/RSK pathway” as used here and in the claims, shall mean the sequence of biochemical events that is initiated when RANTES or a RANTES-related chemokine binds to a high affinity receptor on a parenchymal cell of the CNS, for example, to an astrocyte or to a glial cell. The biochemical events of the RANTES/RSK pathway include a series of phosphorylations by specific protein kinases, including MEK, ERK, and RSK. As a result of RSK phosphorylation, tranlocation of phosphorylated RSK into the nucleus occurs, causing up-regulation of genes encoding various proinflammatory chemokines and cytokines as demonstrated herein.

Structurally, chemokines are a family of small (˜8-14 kDa), basic, related chemoattractant cytokines that play an important role in controlling leukocyte migration (Loetcher, et al., 2000 Adv Immunol 74:127-180). The involvement of chemokines in development of inflammatory, infectious, and autoimmune diseases has been described (Murdoch, et al, 2000. Blood 95:3032-3043; Segerer et al., 2000. J Am Soc Nephrol 11:152-176). Various chemokines including TCA3, MCP-1, MIP-1α, RANTES, and eotaxin (also termed CCL1, CCL2, CCL3, CCL5, and CCL11, respectively) have been associated with autoimmune lesions (Kuchroo et al., 1993. J Immunol 151:4371-4382; Godiska et al., 1995. J Neuroimmunol 58:167-176; Lloyd et al., 1997. J Exo Ned 185L1371-1380). In autoimmune diseases of the central nervous system chemokines expressed in the parenchyma, especially by astrocytes, facilitate the recruitment of inflammatory cells into autoimmune lesions (Sorensen et al., 1999. J Clin Invest 103:807-815; Dorf et al., 2000. J Neuroimmunol 111:109-121; Fischer et al., 2000. J Immunol 167:1637-1643; Luo et al., 2000. J Immunol 165:4015-4023; Matejuk et al., 2000. J Immunol 164:3924-3931; Rajan et al., 2000. J Immunol 164:2120-2130).

The chemokine proteins and glycoproteins are classified into subfamilies based on the position of the first two of four conserved cysteine residues (Zlotnik, et al, 2000. Immunity 12:121-127). Chemokines are divided into four groups known as the CXC, CC, CX3C, and XC chemokines. The CXC subfamily members have a single amino acid residue intervening between the first two-conserved cysteines. The largest collection of chemokines belongs to the CC subfamily in which the first two cysteine residues are adjacent. In the CX3C chemokines three amino acids reside between the first two cysteines. In contrast, members of the XC chemokine family lack one of the first two cysteine residues.

Chemokines interact with specific receptors on the surface of their target cells. All chemokine receptors belong to the superfamily of 7-transmembrane spanning cell surface receptors that are coupled to heterotrimeric guanine nucleotide-binding regulatory proteins (G-proteins; Rossi et al. 2000. Annu Rev Immunol 18:217-242). Generally each chemokine receptor binds multiple chemokines and most chemokines bind to more than one receptor (Loetscher et al., 2000. Adv Immunol 74:127-180; Rossi, et al., 2000. Annu Rev Immunol 18:217-242). RANTES binds to CCR1 and CCR5 receptors on astrocytes (Dorf et al., 2000. J Neuroimmunol 111:109-121).

Embodiments of the invention provided herein include screening methods to identify and obtain inhibitors that are specific to the RANTES/RSK pathway. Sources of chemicals for use in such screening methods include libraries of natural or synthetic products, as are known to those of ordinary skill in the art of chemical screens. Chemical compound libraries can be produced by well known methods, see for example, U.S. Pat. No. 5,908,960 issued Jun. 1, 1999, and WO97/01560, published Jan. 16, 1997.

Inhibitors of the RSK/RANTES pathway provide candidate therapeutic agents capable of alleviating an inflammatory response in the CNS, for example a demyelinating condition such as is found in multiple sclerosis (MS), or post-viral infection or post-treatment with an anti-TNFα agent. Such a therapeutic compound can be prepared as a pharmaceutical composition in a pharmaceutically acceptable buffer, and can contain one or more excipients, and can contain one or more additional therapeutic agents such as a steroid or a non-steroidal anti-flammatory agent.

Modes of systemic administration include, but are not limited to, transdermal, intramuscular, intraperitoneal, intravenous, subcutaneous, intranasal, and oral routes. The compounds may be administered by any convenient route, for example, by infusion or bolus injection, by absorption through epithelial or mucocutaneous linings (e.g., oral mucosa, rectal and intestinal mucosa, etc.), and may be administered together with other biologically active agents.

A pharmaceutically acceptable carrier or excipient can be added. Such a carrier includes but is not limited to saline, buffered saline, dextrose, water, glycerol, ethanol, and combinations thereof. The formulation should suit the mode of administration.

The compositions herein can further comprise wetting or emulsifying agents, or pH buffering agents. The composition can be a liquid solution, suspension, emulsion, tablet, pill, capsule, sustained release formulation, or powder. The compositions can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g., encapsulation in liposomes, microparticles, microcapsules and the like.

A composition herein is formulated in accordance with routine procedures as a pharmaceutical composition adapted for subcutaneous administration to human beings. Typically, compositions for subcutaneous administration are solutions in sterile isotonic aqueous buffer. Where necessary, the composition may also include a solubilizing agent and a local anesthetic to ameliorate pain at the site of the injection. Generally, the ingredients are supplied either separately or mixed together in unit dosage form, for example, as a dry, lyophilized powder or water-free concentrate in a hermetically sealed container such as an ampoule or sachette, for example, indicating the quantity of active agent. Where the composition is to be administered by infusion, it can be dispensed with an infusion bottle containing sterile pharmaceutical grade water, buffer, or saline. Where the composition is administered by injection, an ampoule of sterile water or saline for injection can be provided so that the ingredients may be mixed prior to administration.

The compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances. Routine determinations of blood levels of an inflammation marker such as TNF-α are made by one of ordinary skill in the art. However, suitable dosage ranges for subcutaneous administration are generally about 20-500 micrograms of each active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

The invention in other embodiments provides a pharmaceutical pack or kit comprising one or more containers filled with one or more of the ingredients of the pharmaceutical compositions of the invention. Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

Compositions herein can be combined with other agents, for example, with antibiotics, antivirals, anti-inflammatory agents, anti-convulsives, and other compositions known to one of ordinary skill in the pharmaceutical arts. For example, a neurodegenerative or autoimmune disease may be treated with a composition as described herein in combination with an agent such as β-interferon, or with a random copolymer comprising amino acids such as Copaxone®, or a composition as described in Fridkis-Hareli et al. 2002, J Clin. Invest. 109: 1635.

The invention in various embodiments now having been fully described, additional embodiments are exemplified by the following Examples and claims, which are not intended to be construed as further limiting. The contents of all cited references are hereby incorporated by reference herein.

### EXAMPLES

The following materials and methods are used throughout the examples herein.

Materials and Methods

Mice. BALB/cJ mice were purchased from Jackson Laboratories (Bar Harbor, Me) and were bred in animal facilities. CCRI and CCR3 knockout mice were bred on a mixed 129/BALB background (Gerard, et al., 1997. J Clin Invest 100:2022-2027; Shi, et al., 2000. J Clin Invest 105:945-953), while CCR2 and CCR5 deficient mice were on a mixed 129/C57BL background (Sato, et al., 1999. J Immunol 163:5519-5525). Mice were maintained in accordance with the guidelines of the Committee on Animals of the Harvard Medical School.

Reagents. Recombinant derived mouse TNFα, RANTES, eotaxin, MIP-1α, MIP-1β, and SDF-1α were purchased from R&D System (Minneapolis, Minn.). Recombinant murine IL-1β was purchased from Invitrogen (Carlsbad, Calif.). U0126 was purchased from Cell signaling Technology (Beverly, Mass.) while SB203580 was purchased from Calbiochem (San Diego, Calif.). Rabbit antibodies directed to p44/p42 MAP kinase (ERK1/2), phospho-p44/p42 MAP kinase (Thr202/Tyr204) (P-ERK1/2), phospho-MEK1/2 (Ser217/221), phospho-SAPK/JNK (Thr183/Tyr185), phospho-p38 MAP kinase (Thr180/Tyr182), p90RSK, phospho-p90RSK (Ser381), phospho-p90RSK (Thr360/Ser364), and phospho-p90RSK (Thr574) were all purchased from Cell Signaling Technology (Beverly, Mass.). Hoechst 33258 was purchased from Sigma Chemicals Co. (St. Louis, Mo.).

Recombinant mouse MCP-1, anti-MCP-1 Ab, biotin labeled anti-MCP-1 Ab, anti-TNFα Ab, biotin labeled anti-TNFα Ab, and anti-ICAM-1 Ab were purchased from BD-PharMingen (San Diego, Calif.). TCA4 was prepared as described in Tanabe et al., 1997. J Immunol 159:5671-5679. Anti-CX3CR1 Ab was purchased from Torrey Pines Biolabs, Houston, Tex. These reagents were treated with Detoxi-Gel™ (Pierce Chemical Co., Rockford, Ill.) to minimize potentially remaining endotoxin prior to use. LPS and ConA were obtained from Sigma Chemical Co., St. Louis, Mo. Pertussis toxin was purchased from List Biological Labs, Campbell, Calif.

H-89, protein kinase A inhibitor 14-22 amide, Rp-8-bromo-cAMP, 8-bromo-cAMP, dibutyryl cAMP (db-cAMP), forskolin, pertussis toxin (PTx), Raf-1 inhibitor I, SB203580 and GF109203 were purchased from Calbiochem (San Diego, Calif.).

Astrocyte isolation and culture. Astrocytes were prepared from neonatal (less than 24 h) mouse brains, as described in Luo, Y., et al. 2000. J Immunol 165:4015. The purity of astrocyte cultures was greater than 95%, as determined by indirect immunofluorescence with anti-glial fibrillary acidic protein antibodies, with anti-Mac-1 to detect microglial cells, and anti-galactocerebroside to detect oligodendrocyte contamination.

ELISA. For production of supernatants, 2×10⁴ astrocytes were cultured in 96 well plates with medium or 100 ng/ml RANTES. Supernatants were collected after the indicated times. ELISA assays for KC and MCP-1 were performed as in Luo, Y., et al. 1999. J Immunol 163:3985, and for TNF-α as in Abromson-Leeman, et al. 2001. Eur J Immunol 31:527. Protein levels were determined using recombinant KC, MCP-1, or TNF-α (R & D Systems, Minneapolis, Minn.) as standards.

SDS-PAGE and Western blotting. Astrocytes were treated for the indicated time with media or 100 ng/ml RANTES, eotaxin, or other materials as indicated. Cells (3×10⁵ in Examples 1-3, or 2×10⁴ in later Examples) were resuspended in 100 μl buffer (50 mM HEPES, pH 7.5, 150 mM NaCl, 2 mM EDTA, 2 mM EGTA, 1% Triton X-100, 50 mM Sodium fluoride, 5 mM Sodium pyrophosphate, 50 mM Sodium β-glycerophosphate, 1 mM Sodium Ortho-vanadate, 1 mM DTT, 1 mM PMSF, 10 μg/ml Leupeptin, and 10 μg/ml Aprotinin). Protein concentration of the whole cell extract was determined by BCA protein assay kit (Pierce, Rockford, Ill.). Samples (10 μg) were loaded and separated on a 10% SDS-polyacrylamide gel. After transfer to Hybond ECL nitrocellulose membrane (Amersham Pharmacia Biotech Inc, Piscataway, N.J.) or Zeta-probe blotting membranes (Bio-Rad, Richmond Calif.) blots were blocked overnight with 5% BSA at 4° C., and probed with the antibody indicated. Antibody against unphosphorylated or phosphorylated erk (Cell Signaling, Beverly, Mass.) was used at 1 μg/ml. Appropriate anti-immunoglobulin reagents were used to develop the blots by enhanced chemiluminescence (Amersham Pharmacia Biotech Inc, Piscataway, N.J.).

RNA isolation and RNase protection assay (RPA). RNA was prepared as in Luo, Y., et al. 1999. J Immunol 163:3985. RNase protection assays (RPA) for chemokine message were conducted with multi-probe templates according to the manufacture's protocol (RiboQuant assay kit, BD-PharMingen, San Diego, Calif.). Gels were scanned and radioactive bands quantitated using a phosphoimager (Molecular Dynamics, Sunnyvale, Calif.). Levels of uniformly expressed housekeeping genes large ribosomal subunit protein 32-3A (L32) or glyceraldehyde-3-phosphate dehydrogenase (GAPDH), were used for normalization. The value of each chemokine receptor band divided by the value of the indicated housekeeping gene band in the same sample yielded the relative intensity. Normalized receptor expression represents the ratio of the relative intensity following treatment with chemokine versus the relative intensity of medium alone.

Plasmids, transient transfection, and luciferase activity assay. The KC reporter plasmid was constructed by using a luciferase reporter gene pGL-3 basic vector (Promega, Madison, Wis.) driven by mouse KC promoter (−2878/+43). Wild-type p90RSK expression (WT pKH3) and dominant negative p90RSK expression (ΔΔ RSK pKH3) plasmids were obtained from Dr. John Blenis, Harvard Medical School. Astrocytes were transiently transfected with Lipofectamine 2000 reagent (Life Technologies, Gaithersburg, Md.) according to the manufacturer's protocol. After 24 h, the cells were stimulated with RANTES in absence or presence of the indicated inhibitor, and luciferase activity was determined after an additional 24 h, by the procedure according to the manufacturer (Promega, Madison, Wis.). Relative luciferase activity was normalized for cell lysate protein concentration as determined by BCA protein assay kit (Pierce, Rockford, Ill.). The Relative Fold Induction is the relative intensity of the experimental sample divided by the relative intensity of the medium control.

Immunofluorescence. Astrocytes were grown on glass coverslips for a day; the cells were then serum-starved for three hours. Astrocytes were treated with RANTES for 5 or 20 minutes. Following treatment, cells were fixed in −20° C. methanol, permeabilized in ice-cold 0.2% Triton X-100 in PBS and incubated with phospho-p90RSK (Thr360/Ser364) antibody overnight at 4° C., followed by incubation with Alexa Fluor 555 goat anti-rabbit IgG (Molecular Probes, Eugene, Oreg.). Nuclei were stained with Hoechst 33258 (1:2000) for 5 min; coverslips were mounted on slides using the ProLong Antifade Kit (Molecular Probes, Eugene, Oreg.). Slides were stored at room temperature in the dark until observation.

Flow cytometric analyses. PBS with 0.1% sodium azide was used for all washes and antibody (Ab) dilutions. Cells were pre-incubated 15 min with 0.5 μg Fc Block (BD-PharMingen) then 40 min at 4° C. with 1 μg of the appropriate Ab or matched isotype control immunoglobulin. Fluorescence-labelled cells were analyzed on a Coulter Profile II flow cytometer (Coulter, Hialeah, Fla.).

Statistics. Except where noted all experiments were performed on at least 3 separate occasions. Numerical data are presented as the mean±SEM. Statistical analysis was performed with Student's t-test. P values less than 0.01 were considered significant.

RT-PCR. Total RNA and cDNA were prepared from ≧3×10⁵ astrocytes as detailed elsewhere (Tanabe et al., 1997a. J Neurosci 17:6522-6528). The sequences of the KC primers were GCGAATTCACCATGATCCCAGCCACCCG (SEQ ID NO: 1) and GCTCTAGATTACTTGGGGACACCTTTTAG (SEQ ID NO: 2); and the β-glucuronidase primers were ATCCGAGGGAAAGGCTTCGAC (SEQ ID NO: 3) and GAGCAGAGGAAGGCTCATTGG (SEQ ID NO: 4). The primer pairs were designed to span an intron. PCR was carried out in a 20 μl reaction mixture with 0.4 μl cDNA, 0.5 μM of each primer, and the manufacturer's Taq DNA polymerase conditions (Qiagen Inc., Valencia, Calif.). The PCR program included preincubation at 94° C. for 2 min, amplification for 27-30 cycles of PCR at 94° C. for 50 sec plus 55° C. -58° C. annealing for 50 seconds plus 72° C. extension for 50 sec, and a final 72° C. 10 minute extension. Six microliters of the PCR mixtures were visualized on 3% agarose minigels.

Treatment with pharmacological inhibitors. Astrocytes were washed and resuspended in serum-and insulin-free complete medium and starved overnight. Cells were treated at 37° C. for 1 h with 100 ng/ml Pertussis toxin (PTx) before addition of RANTES or IL-1. Genistein, wortmannin, or U0126 were added 30 min prior to addition of chemokine. The cells were harvested after a 3 h incubation with RANTES; RNA was prepared and assayed by RPA. The viability of the cells with or without inhibitors was greater than 95%.

PKA activity assay. PKA activity was determined using a commercially available kit (Calbiochem, San Diego, Calif.) according to manufacturer's recommendations. Primary mouse astrocytes were grown in 6-well plates and then stimulated as described and lysed in 100 μl buffer (same buffer used for Western blotting) for 30 min. Five μl of the lysates were incubated with 20 μl PKA reaction mixture at 30° C. for 30 min. The reaction was terminated by adding 10 μl stop solution and ³²P radioactivity was counted. Biotinylated Kemptide (LRRASLG; SEQ ID No:5) was used as a highly specific substrate for assessment of PKA activity.

### Example 1

### RANTES Stimulation of Astrocytes Induces KC and TNF-α Synthesis

Previous reports (Luo, Y., et al. 2000. J Immunol 165:4015; McManus, C. M., et al. 2000. Am J Pathol 156:1441; Han, Y., et al. 2001. J Clin Invest 108:425) indicate that chemokines such as the CXC-chemokines KC, MIP-2, and SDF-1α, and the CC-chemokines MIP-1α and MIP-1β induce chemokine/cytokine amplification, however those reports failed to examine RANTES.

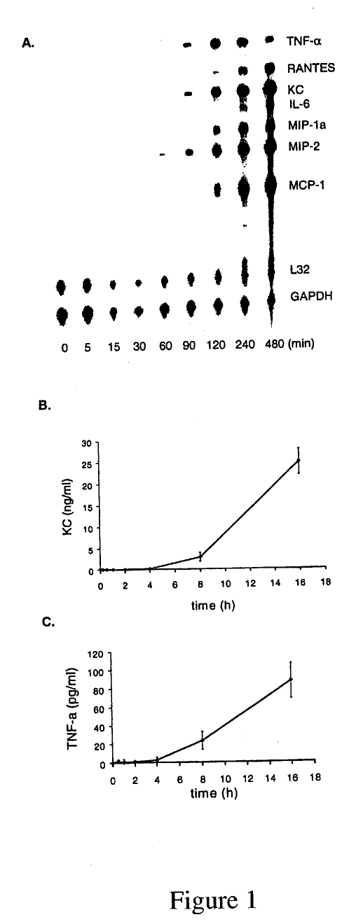

The ability of RANTES to stimulate chemokine/cytokine transcripts in mouse astrocytes was examined by RNase Protection Assay (RPA; FIG. 1). Primary neonatal mouse astrocyte cultures were incubated with medium or with RANTES for the indicated times and then harvested for RNA isolation. Preliminary experiments indicated that 100 ng/ml RANTES yielded optimal stimulation. Following RANTES treatment, the expression of TNF-α, RANTES, KC, IL-6, MIP-1α, MIP-2, and MCP-1 transcripts was up-regulated (FIG. 1 panel A). TNF-α, KC, and MIP-2 transcripts were detected as early as 60-90 min after RANTES stimulation. At 2 h, distinct bands for MIP-1α, and MCP-1 became visible. RANTES and IL-6 transcripts were the last to appear (4-8 h). Untreated control astrocytes expressed message for the housekeeping genes L32 and glyceraldehyde-3-phosphate dehydrogenase (GAPDH) and occasionally trace levels of RANTES or MCP-1.

The ability of RANTES to stimulate KC and TNF-α protein synthesis was next examined. Primary astrocyte cultures were treated with 100 ng/ml RANTES for the indicated times, then supernatants were harvested for assay. KC protein synthesis was detectable within 8 h and the level rose rapidly (FIG. 1 panel B). TNF-α protein was induced with similar kinetics. Low levels of TNF-α (less than 120 pg/ml) were detectable after 16 h compared to the 20-30 ng/ml concentrations of KC (FIG. 1 panels B and panel C). Protein expression of KC and TNF-α was observed at 8 h. TNF-α is a multipotential cytokine involved in microglial activation, neuronal death, and immune regulation (Grunfeld, C., et al. 1990. Adv Intern Med 35:45.; Probert, L., et al. 1997. J Neuroimmunol 72:137). Thus, the early release of KC and TNF-α during an inflammatory response have pathological consequences.

It is here demonstrated that RANTES stimulates murine astrocytes to synthesize RNA for multiple chemokines including KC, MIP-2, MIP-1α, MCP-1, and RANTES plus the cytokines TNF-α and IL-6. This in vitro model reflects the complex pattern of chemokines and cytokines produced by astrocytes during chronic infectious and inflammatory diseases (Huang, D., et al. 2000. Immunol Rev 177:52; Luo, Y., et al. 2000. J Immunol 165:4015; Fischer, F. R., et al. 2000. J Neuroimmunol 110:195; 1998). The ability of one chemokine to induce a cascade of proinflammatory mediators represents an amplification mechanism that may prolong inflammatory responses in the CNS.

Sixty to 90 min after stimulation, KC, MIP-2, and TNF-α transcripts were up-regulated, suggesting that these genes can be classified as an “immediate early genes” with respect to response to the proinflammatory mediator RANTES. The strong, rapid, and sustained induction of KC indicate an important role for this chemokine. Without being limited by any particular mechanism, KC and the structurally related chemokine MIP-2 can contribute to inflammation by recruiting leukocytes to the CNS, and to subsequent repair processes by promoting the growth of oligodendrocytes (Wu, Q., et al. 2000. J Neurosci 20:2609).

### Example 2

### RANTES Activates the MAP Kinase Pathway in Astrocytes

Little is known about the signaling mechanisms involved in astrocyte respones to chemokines. To examine components of the RANTES signal transduction pathway, phosphorylation of the MAP kinase kinase (MEK) and mitogen activated protein (MAP) kinases was examined. To minimize basal kinase activity, astrocytes were starved of serum for at least 3 h before treatment with chemokine. Whole cell lysates were separated by SDS-PAGE and examined by Western blot. Antibodies that specifically react with phosphorylated MEK or phosphorylated extracellular signal-related kinase (ERK) were used to detect the active kinases.

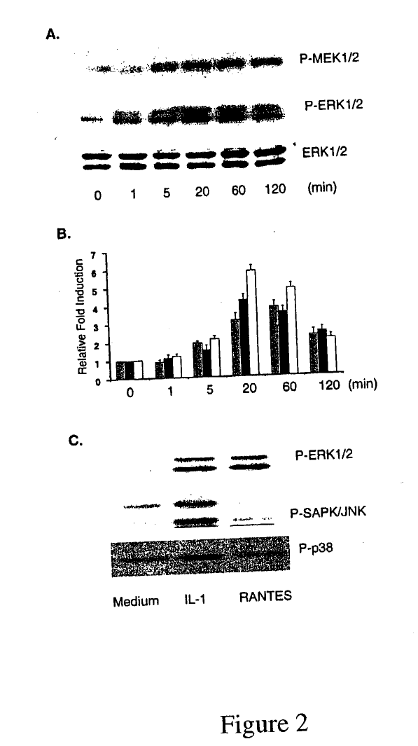

RANTES-induced phosphorylation of MEK, ERK1 and ERK2 was observed to appear within 5 min. Phosphorylation peaked at 20-60 min, and lasted for over 2 h (FIGS. 2 panels A, B). For normalization, total ERK protein levels were determined with an antibody (Ab) that reacts both with the phosphorylated and non-phosphorylated proteins.

To determine whether RANTES also induced phosphorylation of the p38 and SAPK/JNK MAP kinases, the state of these enzymes was examined directly by Western blot. As shown in FIG. 2 panel C, RANTES treatment failed to phosphorylate p38 or SAPK/JNK. Kinetic studies indicated that phosphorylation of p38 or JNK was not detectable from 5 to 120 min after RANTES stimulation. Furthermore, treatment with the p38 inhibitor SB203580 failed to modulate RANTES-mediated transcription of chemokine RNA. All three MAP kinases were activated following treatment with the cytokine IL-1 (FIG. 2 panel C).

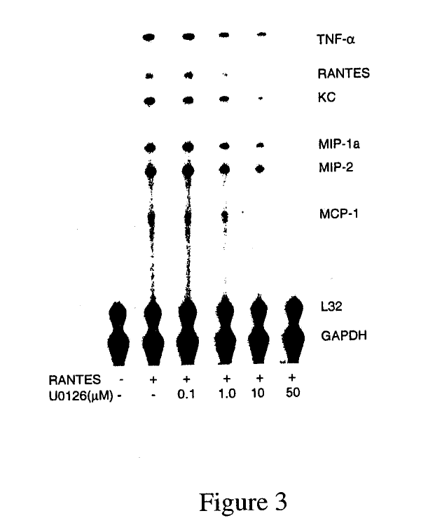

To examine the effects of the MAP kinase pathway on induction of chemokine transcripts, astrocyte cultures were pretreated with the specific MEK1/2 inhibitor, U0126, for 1 h. Treatment with U0126 at 1 μM partially inhibited transcription, and incubation with 50 μM completely inhibited TNF-α and chemokine transcription (FIG. 3). The data indicate that MEK is involved in the intracellular signal required for RANTES-mediated chemokine induction of astrocytes.

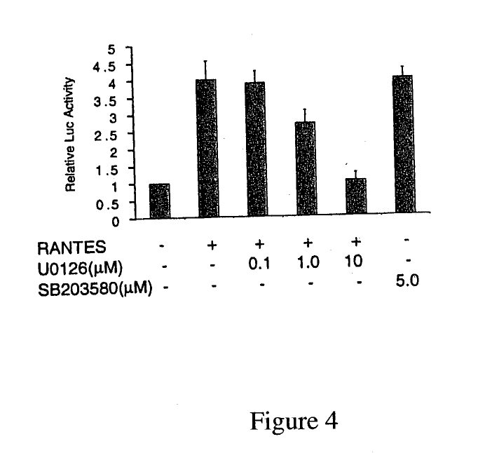

To establish whether RANTES up-regulation of chemokine production is mediated by activation of promoter elements, primary astrocyte cultures were transiently transfected with a murine KC promoter-luciferase construct, and reporter activity was monitored after RANTES treatment. RANTES was found to stimulate reporter activity by about four fold. Treatment of the transfected cells with U0126 was found to inhibit reporter activity, while incubation with SB203580, an inhibitor of p38 MAP kinase, did not inhibit the reporter (FIG. 4).

In a system that analyzed the growth and differentiation of human first trimester fetal astrocytes, it was shown that RANTES induced nuclear translocation of STAT-1 proteins (Bakhiet, M. 2001. Nat Cell Biol 3:150). However, preliminary experiments with mouse neonatal astrocytes suggest that STAT-1 does not play a role in RANTES-mediated chemokine synthesis. The Examples herein show that these different RANTES-mediated effector capacities involve separate elements for nuclear translocation and transcriptional activation.

Many proinflammatory mediators are known to induce expression of CXC chemokines belonging to the family of KC related gene products (e.g. GRO, IL-8, CINC). In various cell types, induction of these chemokine ligands includes participation of the ERK and p38 MAP kinases (Krause, A., et al. 1998. J Biol Chem 273:23681; Holtmann, H., et al. 1999. Mol Cell Biol 19:6742; Bian, Z. M., et al. 2001. Exp Eye Res 73:111). In contrast, in the present system KC induction appears to be independent of p38 signaling (FIG. 4), indicating that different signaling pathways regulate gene expression in astrocytes.

### Example 3

### RANTES Stimulates Activation of RSK in Astrocytes.

The p90 ribosomal S6 protein kinases (RSKs) are a family of Ser/Thr protein kinases that are stimulated through the ERK pathway (Frodin, M., et al. 1999. Mol Cell Endocrinol 151:65). Further, RSKs participate in the regulation of transcription factors such as CREB, CREB-binding protein and p300, and c-Fos (Xing, J., et al. 1996. Science 273:959; Nakajima, T., et al. 1996. Cell 86:465; Fisher, T. L., et al. 1996. Mol Cell Biol 16.1212). In addition, RSK participates in the phosphorylation of IκB leading to the activation and nuclear translocation of NFκB (Schouten, G. J., et al. 1997. Embo J 16:3133; Ghouda, L., et al. 1997. J Biol Cem 272:21281). Activation of RSK is a step-wise process involving phosphorylation of multiple residues.

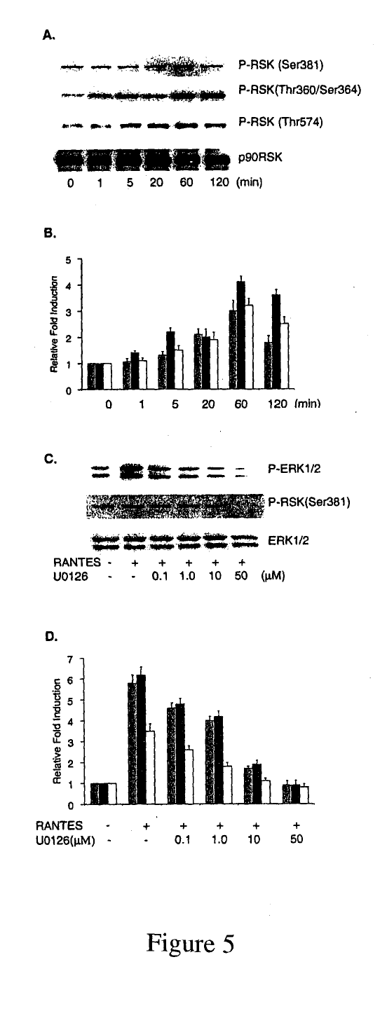

To determine whether RANTES can mediate RSK activation in astrocytes, cells were treated with 100 ng/ml RANTES, and phosphorylation of multiple RSK residues was examined by Western blot (FIG. 5 panel A). Increased phosphorylation was observed with all phospho-RSK Ab examined, including reagents specific for phosphorylation at residues Ser381, Thr360/Ser364, and Thr574. Phosphorylation of RSK appeared within 5 min, peaked at 60 min, and was sustained for over 2 h.

To establish the relationship between MEK and RSK, astrocytes were treated with varying concentrations of MEK inhibitor U0126 before stimulation with RANTES. After 20 min, lysates were prepared, and were examined for ERK and RSK phosphorylation. As indicated in FIGS. 5 panels C and D, treatment with the MEK inhibitor was found to block RSK phosphorylation in a dose dependent fashion. These results show that MEK is an upstream kinase responsible for activation of RSK in the RANTES signal transduction pathway.

### Example 4

### Effect of a Dominant Negative RSK Mutant on KC Trancription

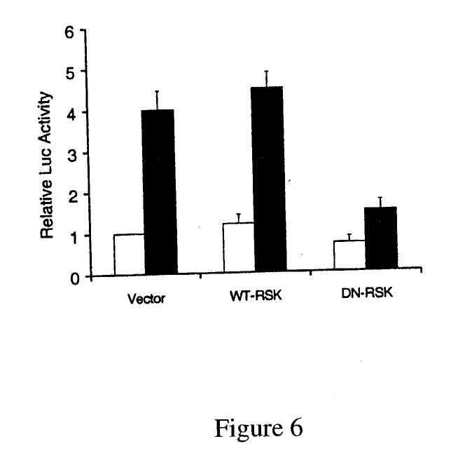

To examine the RSK dependence of RANTES-stimulated activation of the KC promoter, a dominant negative mutant of RSK was employed. The two phosphorylation sites in RSK (K112/464R) required for kinase activity were mutated, resulting in a kinase defective protein (Ghoda, L., et al. 1997. J Biol Chem 272:21281; Kwon, E. M., et al. 2000. Blood 95:2552). Astrocytes were cotransfected with the luciferase-KC promoter construct along with a gene for either wild type RSK, the mutant RSK, or a vector control. The cotransfected cells were stimulated with RANTES and were monitored for luciferase reporter activity. Dominant negative RSK was found herein to specifically suppress reporter activity (FIG. 6), demonstrating the importance of this enzyme in regulating the transcription of the gene encoding chemokine KC.

### Example 5

### RANTES Induces Nuclear Translocation of RKS.

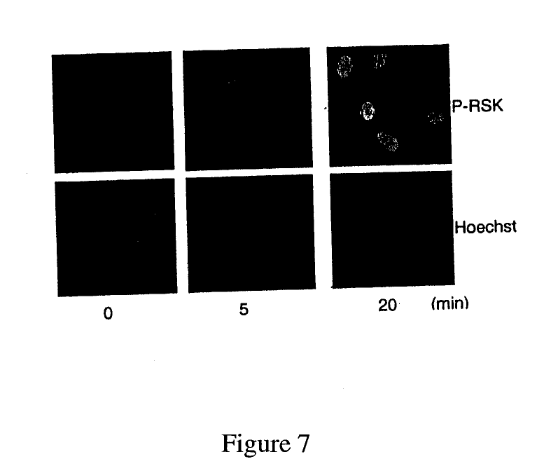

The ability of RANTES to induce nuclear translocation of RSK was examined. While untreated astrocytes were found to display a diffuse distribution of phosphorylated RSK, nuclear translocation was noted at 5 min after RANTES treatment, and after 20 min high levels of phosphorylated RSK were found to be concentrated within the nucleus (FIG. 7).

Phosphorylation and translocation of RSK are novel requirements for RANTES mediated activation of chemokine synthesis in astrocytes. Because the ERK-RSK signal transduction pathway used by astrocytes is distinct from the reported mechanisms of chemokine signaling and induction utilized by leukocytes, then therapeutic strategies to regulate chemokine responses and synthesis can be directed to specific target tissues, i.e., tissues arising from astrocytes in the CNS.

The data show that RANTES activates the Ser/Thr kinase, RSK, downstream of ERK. In addition, RANTES stimulation causes translocation of phosphorylated RSK to the nucleus. Transfection of a dominant negative RSK mutant that lacks kinase activity specifically inhibited KC promoter driven transcription. The kinase-defective RSK inhibited but did not completely abolish RANTES-induced transcription, suggesting that other signaling pathways may be involved in transcriptional activation of KC. Evidence herein shows that RSK is involved in signaling pathways in astrocytes, and that RSK is involved in chemokine signaling.

Recent reports noted involvement of MEK and ERK1/2 in response to SDF-1 (Han, Y., et al. 2001. J Clin Invest 108:425; Lazarini, F., et al. 2000. Eur J Neurosci 12:117; Bajetto, A., 2001. J Neurochem 77:1226). It is demonstrated herein that these enzymes are also involved in astrocyte signal transduction following RANTES treatment.

In contrast, RANTES induces activation of the p38 MAP kinase pathway in T cells as evidenced by the rapid RANTES-dependent phosphorylation and activation of p38 MAP kinase as well as the activation of its downstream effector MAP kinase-activated protein (MAPKAP) kinase-2 (Wong, M., 2001. J Biol Chem 276:11427). Pharmacological inhibition of RANTES-dependent p38 MAP kinase activation blocks MAPKAP kinase-2 activity in T cells (Wong, M., 2001. J Biol Chem 276:11427). Examples herein show that RANTES differentially activates distinct MAP kinases in a cell type specific fashion, i.e., activates astrocytes differently than lymphocytes.

### Example 6

### Treatment of Astrocytes with RANTES or Eotaxin Induces Chemokines.

The ability of TNFα, eotaxin, RANTES, or TCA4 to up-regulate expression of transcripts for the chemokine KC was evaluated in cultured mouse astrocytes. KC is associated with inflammatory lesions in experimental allergic encephalomyelitis (EAE; Fischer et al., 2000. J Neuroimmunol 110:195-208; Luo et al., 2000. J Immunol 165:4015-4023), and is a potent promoter of oligodendrocyte precursor proliferation (Robinson et al., 1998. J Neurosci 18:10457-10463).

Treatment of astrocytes with RANTES or cotaxin was found to induce synthesis of KC message. (see Luo, et al 2002. Glia 39, 19-30, the contents of which are hereby incorporated by reference). The specificity of these responses was demonstrated by the failure of TCA4 or medium to induce KC. Stimulation with TNF-α was used as a positive control. Additional controls include examination of all samples for the housekeeping gene β-glucuronidase, expression of which was not affected by these treatments.

The ability of RANTES or eotaxin to stimulate chemokine protein synthesis was also examined. Primary astrocyte cultures were treated with 1.5 to 100 ng/ml RANTES, eotaxin, or TCA4 for 48 h, then supernatants were harvested for assay. Incubation with ≧10 ng/ml RANTES or ≧25 ng/ml eotaxin induced KC protein. However, incubation of astrocytes with up to 100 ng/ml TCA4 failed to induce significant levels of KC protein. The kinetics of each of RANTES- and eotaxin-induced KC expression was also examined. KC protein was detected 12 h after stimulation and reached a plateau 1 day after incubation with 100 ng/ml RANTES. The inability of TCA4 to stimulate KC synthesis at all time points confirmed the specificity of these responses to a set of agents identified herein as RANTES-related chemokines, e.g., RANTES and eotaxin.

The ability of RANTES and RANTES-related chemokines to stimulate chemokine/cytokine transcripts in mouse astrocytes was examined by RPA. Primary BALB/cJ neonatal astrocyte cultures were incubated with medium or chemokine for 6 h and then harvested for RNA isolation. Untreated astrocytes expressed message for the housekeeping genes L32 and GAPDH and occasionally traces of RANTES or MCP-1. Following treatment of astrocytes with RANTES, eotaxin, MIP-1α, or MIP-1 β the expression of TNF-α, RANTES, KC, MIP-1α, MIP-2, and MCP-1 transcripts were up-regulated. In separate experiments IP-10 transcripts were also detected. Maximal levels of mRNA were noted following stimulation with greater than or equal to 100 ng/ml RANTES or eotaxin. MIP-1α and MIP-1β also induced RNA synthesis, but the activity of these chemokines was variable. In contrast, treatment with the CC-chemokines MCP-1 and TCA4 or the CXC-chemokine SDF-1α had no effect on RNA expression.

To ascertain whether endotoxin might have contributed to the activity of RANTES and eotaxin, the chemokines were boiled for 30 min before incubation with astrocytes. Boiling completely destroyed the ability to induce chemokine transcripts, suggesting that the activity was attributable to chemokine and not to endotoxin which is stable under these experimental conditions. To investigate synthesis of additional chemokine proteins, expression of MCP-1 in culture supernatants was evaluated. MCP-1 proteins were detected following stimulation with 12 ng/ml to 25 ng/ml RANTES or eotaxin, but not with 100 ng/ml TCA4. MCP-1 proteins were detectable 6 h after chemokine stimulation.

The kinetics of RANTES-induced chemokine production shows that TNF-α mRNA was detected after 1.5 h but disappeared after 18 h. At 3 h bands for RANTES and MIP-1α were visible. RANTES transcription was sustained for greater than 24 h while MIP-1α mRNA was already down regulated by 24 h. Transient production of IL-6 transcripts was noted at 6-24 h. Transcripts for the chemokine TCA3 were not detected at any time point. To establish that RANTES also induced cytokine protein production in astrocytes, TNF-α protein levels were measured. TNF-α protein was not detectable in culture supernatants 6 h after RANTES stimulation, but greater than 100 pg/ml of TNF-α were detected 12 h after stimulation and reached peak levels at 24 h. The quantity of TNF-A released could contribute to a self-limiting feedback loop capable of prolonging astrocyte activation.

To evaluate the cellular specificity of chemokine induction by RANTES-related chemokines, mouse thymocytes were treated with 2.5 μg/ml ConA, 100 ng/ml RANTES, or TCA4. After 6 h the cells were harvested for RNA extraction. Thymocytes expressed background levels of RANTES and TNF-α RNA, and these levels were found not to ber further enhanced following treatment with these chemokines. However, Con A treatment consistently up-regulated MIP-1α and TNF-α transcripts. Thus, the ability of RANTES-related chemokines to up-regulate chemokine/cytokine synthesis is not a generalized phenomenon in all cell types, but is specific for CNS cells such as astrocytes.

### Example 7

### Astrocyte Responses to RANTES and Eotaxin are Sensitive to Pertussis Toxin

High affinity RANTES receptors include CCR1, CCR3, and CCR5 (Gao, et al., 1995. j Biol Chem 270:17494-17501; Post et al., 1995. J Immunol 164:2120-2130; Boring et al., 1996. J Biol Chem 271:7551-7558) while those for eotaxin include CCR2, CCR3, and possibly CCR5 (Daugherty et al., 1996. J exp Med 183:2349-2354; Ponath et al., 1996. J Exp Med 183:2437-2448; Ogilvie et al., 2001. Blood 97:1920-1924). CCR1 and CCR5 are expressed on mouse astrocytes (Tanabe et al., 1997a. J Immunol 159:5671-5679; Dorf et al., 2000. J Neuroimmunol 111:109-121; Han et al., 2000. Glia 30:1-10). In contrast, expression of CCR2 and CCR3 message was not detected by RT-PCR in mouse astrocytes (Heesen et al., 1996. J Neurosci Res 45:382-391; Dorf et al., 2000. J Neuroimmunol 111:109-121).

CCR1 and CCR5 are coupled to G proteins often of the Gαi class. Since Gαi functions are specifically inhibited by Pertussis toxin (PTx) astrocytes were treated with 100 ng/ml PTx for 1 h before stimulation with RANTES, eotaxin or IL-1β. Culture supernatants were examined for the presence of MCP-1 protein. PTx was found to specifically inhibit both RANTES and eotaxin induced chemokine synthesis, but did not diminish IL-1 induced MCP-1 levels. Since PTx inhibition was partial, the findings suggest that both PTx sensitive and resistant G proteins participate in RANTES-mediated astrocyte signaling.

To determine which G protein coupled chemokine receptor was responsible for RANTES or eotaxin responsiveness, astrocytes from mice genetically deficient for CCR1, CCR2, CCR3, or CCR5 were incubated with 100 ng/ml RANTES, eotaxin, or TCA4. The data showed that astrocytes derived from each donor specifically responded to both RANTES and eotaxin by production of chemokine transcripts. The responses to these chemokines were thus not uniquely dependent on any single receptor.

In cells used for these examples, CCR1 and CCR3 astrocyte cultures were derived from adult mice, while other astrocyte cultures were of neonatal origin. Thus, the ability of RANTES or cotaxin to stimulate chemokine transcripts was found to be a general characteristic of astrocytes, and is not dependent on the age of the cell donor.

### Example 8

### Chemokine Mediated Signaling.

RANTES treatment of astrocytes stimulates the MAP kinase (MAPK) pathway as shown by phosphorylation of the erk1/erk2 proteins within 5 to 20 min. To demonstrate equivalent loading in each lane, total levels of erk proteins were compared. To assess the role of the MAPK pathway in chemokine production, astrocytes were treated with the MEK inhibitor U0126 (50 μM), 100 μM genistein (a protein tyrosine kinase inhibitor) or 1 μM wortmannin (an irreversible inhibitor of phosphatidylinositol 3-kinase).

Genistein treatment was found herein to block chemokine message. Addition of the MEK inhibitor U0126 also blocked induction of chemokine transcripts. Although PI-3 kinase activation can stimulate erk1/erk2 phosphorylation (Lopez-Ilasaca et al., 1997. Science 275:394-397), treatment with the PI-3 kinase inhibitor, wortmannin, did not modulate chemokine expression.

### Example 9

### Chemokine-mediated Modulation of Astrocyte Receptors

Astrocytes associated with Alzheimer's Disease, MS and EAE lesions frequently display increased levels of the intracellular marker GFAP, an indicator of astrogliosis (Xu et al., 1999. Glia 25:390-403). To determine if chemokines could modulate GFAP expression astrocytes were treated with 100 ng/ml RANTES for 1-5 days and then examined for the intracellular marker GFAP by conventional immunofluorescence. Evidence for modulation of GFAP was not observed.

Another marker of activated astrocytes in MS and EAE lesions is the increased expression of the adhesion receptor, ICAM-1 (Lee, et al., 1999. J Immunol 165:4658-4666). Control astrocyte cultures were stained with anti-ICAM-1, and RANTES treatment further increased the levels of ICAM-1 protein expression about three-fold (mean intensity 50.2 vs. control of 16.3). In contracst, chemokine TCA4 failed to modulate expression of ICAM-1, demonstrating RANTES-related chemokine specificity.

The cell surface receptor CX3CR1 was also selected for analysis since astrocyte expression of this receptor may facilitate interactions with endothelial cells and neurons that carry the membrane-tethered ligand for this receptor (Bacon, et al, 2000. J Neuroimmunol 104:92-97). Anti-CX3CR1 stained about 50% of the control astrocyte population. Following treatment with RANTES, less than 30% of the cells stained with anti-CX3CR1, and those cells displayed decreased levels of CX3CR1 protein (mean intensity 14.3 vs. control of 6.0). The modulation of receptor proteins peaked at 24 h; similar patterns were noted after a 48 h incubation with RANTES but the intensity levels were intermediate between the 0 and 24 h values.

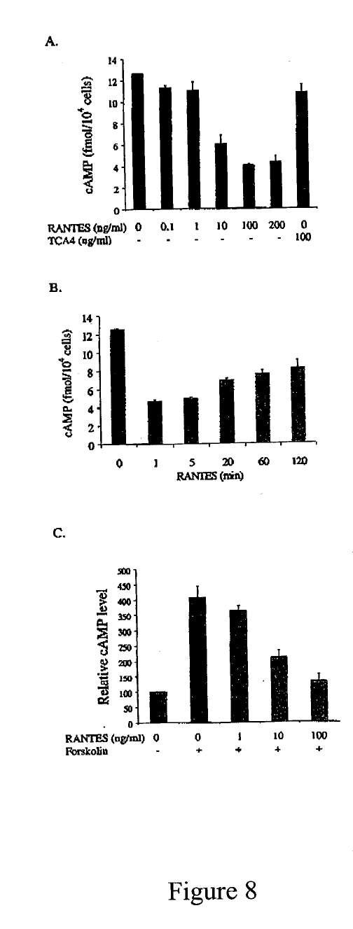

Treatment with the RANTES-related chemokines RANTES or eotaxin, or with TNF-α also found to down-regulate expression of CX3CR1 transcripts. In addition, CXCR4 mRNA levels were decreased, while the levels of CCR1 transcripts were elevated. In contrast, little or no regulation of CCR5 was noted. The pooled results of 3-4 experiments indicate eotaxin caused an average 60% decrease in CXCR4 (P less than 0.01) and a 70% decrease in CX3CR1 mRNA (P less than 0.02) (FIG. 8.B). The effects of RANTES stimulation were similar with an average 70 and 90% reduction in CXCR4 and CX3CR1 transcripts, respectively (P less than 0.01). TNF-α treatment increased CCR1 transcripts by 55% (P less than 0.05) and reduced CX3CR1 and CXCR4 expression (P less than 0.01) with little or no change in CCR5. These effects are specific, as treatment with TCA4 and MCP-1 did not significantly modulate expression of any of the receptor transcripts examined.

The process shown supra of RANTES stimulation of primary neonatal mouse astrocytes induced chemokine and cytokine transcription, includes de novo induction of mRNA for KC, RANTES, MIP-1α, MIP-2, MCP-1, TNF-α and IL-6. This process is initiated through two high affinity RANTES receptors, CCR1 (CC chemokine receptor 1) and CCR5 which are expressed on astrocytes. These 7-transmembrane spanning G protein coupled receptors are often coupled to G proteins that modulate adenylyl cyclase activity (Zhao, et al. 1998. J Cell Biochem 71(1), 36-45).

Without being limited by any particular theory or mechanism, the ability to manipulated RANTES-related chemokines' ability to reorganize surfaces of astrocytes in the CNS may enable the user to affect a broad spectrum of CNS functions in addition to inflammation and neurodegeneration. These functions can include brain development, memory, consciousness, and perception, because of the role of cell surface receptors in interactions of astrocytes with other cell types as glia during development (see Fields, R. et al. 2002 Science 298: 556).

### Example 10

### Decreased Intracellular cAMP Levels After RANTES Stimulation.

To further elucidate the RANTES-mediated signaling pathway in astrocytes, intracellular cAMP levels were evaluated following chemokine stimulation. Primary mouse astrocytes were incubated with the indicated dose of RANTES or the negative control chemokine, TCA4 for 5 min and monitored for cAMP levels. RANTES (100 ng/ml) decreased intracellular cAMP levels by 68% in a dose dependent fashion (FIG. 8A). This response is chemokine specific as another CC-chemokine, TCA4, failed to significantly reduce cAMP levels (FIG. 8A). Kinetic analyses demonstrated that intracellular cAMP was dramatically decreased within 1 min after RANTES stimulation, and slowly recovered as shown at 20 min (FIG. 8A).

Forskolin, an activator of adenylyl cyclase, increased intracellular cAMP levels about 4 fold. RANTES treatment inhibited forskolin-induced cAMP accumulation in a dose dependent manner (FIG. 8A). These data show that RANTES treatment specifically decreases intracellular cAMP levels in astrocytes.

### Example 11

### Effects of RANTES on Astrocytes are Sensitive to Pertussis Toxin (PTx)

Chemokine receptors are generally associated with PTx sensitive Gαi proteins. To examine the PTx sensitivity of RANTES-mediated activation, astrocytes were pretreated with PTx for 1 h, and were stimulated with 100 ng/ml RANTES.

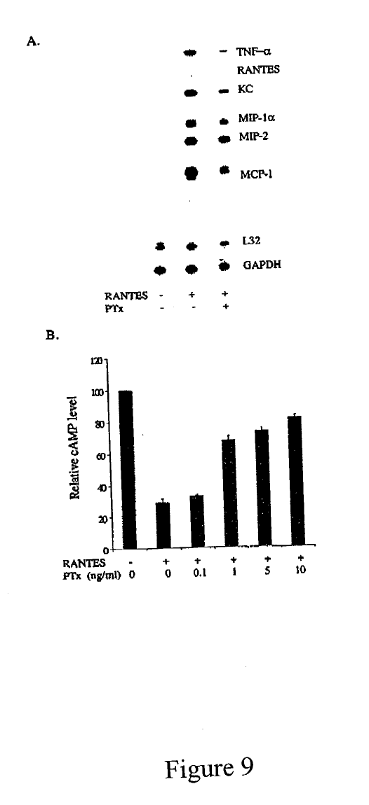

PTx was found to inhibit induction of chemokines RANTES, KC, MIP-1α, MIP-2, MCP-1 and cytokine TNF-α mRNA (FIG. 9A). Inhibition was most pronounced (>50%) for TNFα, KC, MIP-1α and MCP-1. Inhibition of MIP-2 mRNA varied from 23% to 52%. Transcripts for the housekeeping genes L32 and GAPDH were not modified by PTx treatment (FIG. 9A).

PTx also reversed the marked decrease in intracellular cAMP levels following RANTES stimulation (FIG. 9A). The data indicate that RANTES- or RANTES-related chemokine- mediated modulation of cAMP and induction of most proinflammatory mediators are dependent on Gαi proteins.

### Example 12

### Protein Kinase A Activity is Decreased in RANTES Treated Astrocytes

To determine whether RANTES-mediated reduction of cAMP levels affected PKA activity, astrocytes were stimulated with the indicated doses of RANTES for 20 min and were monitored for PKA enzyme activity.

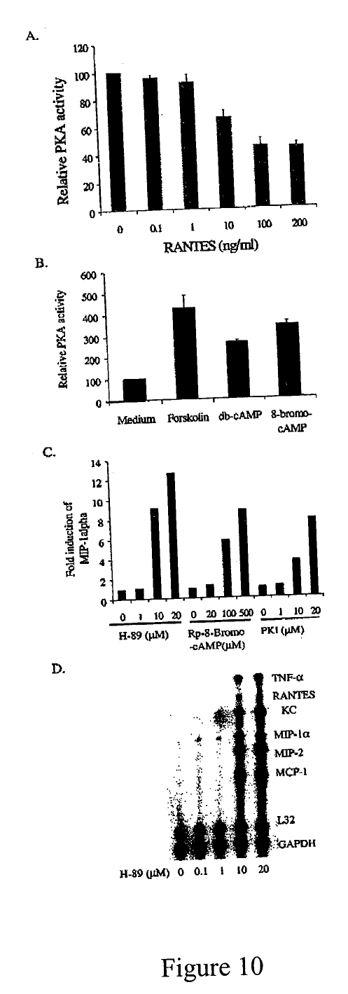

PKA activity was inhibited by 60% following treatment with 100 ng/ml RANTES (FIG. 10A). Kinetic analyses demonstrated kinase activity was maximally reduced 10 min after RANTES stimulation. In contrast, treatment with forskolin or cAMP analogues (db-cAMP and 8-bromo-cAMP) activated astrocyte PKA activity (FIG. 10).

To examine the role of PKA in upregulation of a prototype inflammatory mediator, MIP-1α, three PKA inhibitors: H-89, Rp-8-bromo-cAMP, and PKI (protein kinase A inhibitor 14-22 amide) were employed. All three PKA inhibitors induced expression of transcripts for MIP-1α (FIG. 10C) and other proinflammatory mediators (see for example FIG. 10D). The data demonstrate that inhibition of PKA by RANTES or by pharmacological agents activates astrocytes to produce a series of proinflammatory chemokines and cytokines.

### Example 13

### cAMP Analogues Inhibit Transcription

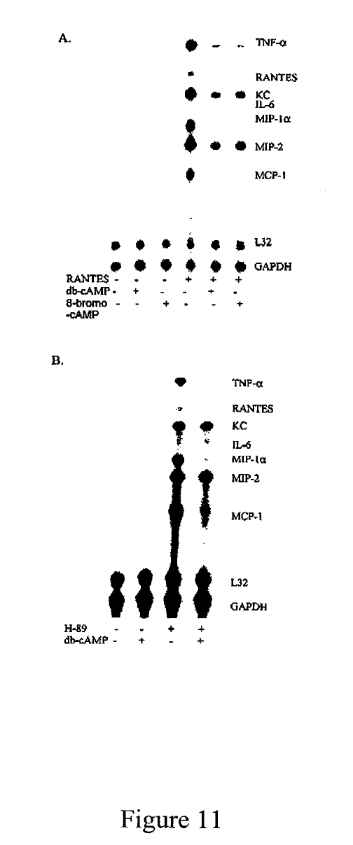

To determine whether there is a link between the effects of decreased cAMP and PKA on transcription of proinflammatory mediators, cAMP analogues db-cAMP and 8-bromo-cAMP were used to determine if these agents could reverse RANTES and H-89-mediated transcription (FIG. 11). Treatment with 500 μM of either of these cAMP analogues was found to inhibit transcription of TNF-α, RANTES, MIP-1α, and MCP-1 by at least 50% (FIG. 11). However, the effects on KC and MIP-2 transcription were weak and transient, peaking at 2 h (FIG. 11A). In contrast, IL-6 mRNA levels were enhanced by 2.0 to 2.4 fold (FIG. 11). Neither cAMP analogue alone had any effect of transcription (FIG. 11A).

These results also show that decreased cAMP and PKA levels are required in astrocytes for transcription of most proinflammatory mediators.

### Example 14

### RANTES Stimulates Activation of Raf-1 in Astrocytes

As shown herein, the MAPK pathway is involved in astrocyte RANTES-mediated chemokine synthesis. To define the signaling elements downstream of PKA and upstream of MEK, Raf-1 activation was examined.

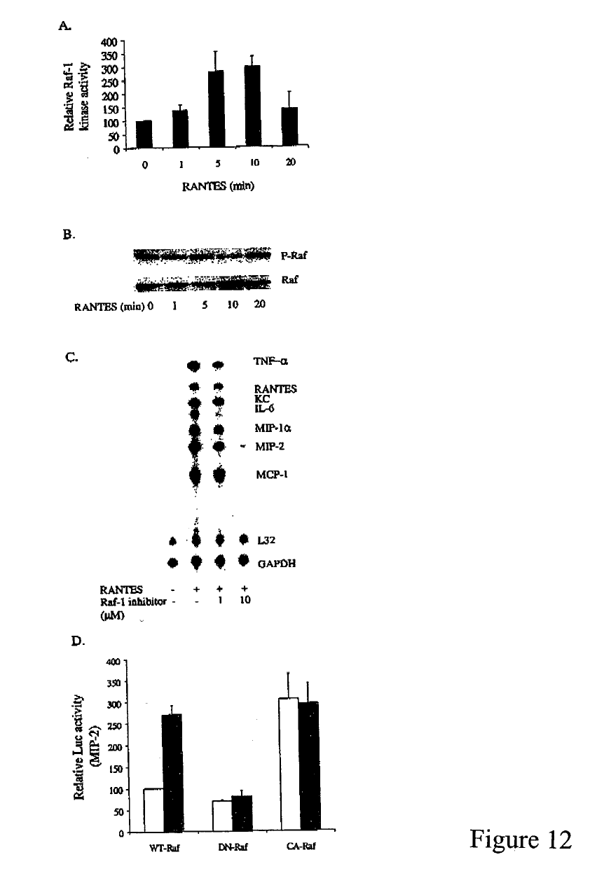

It is herein shown that RANTES induced Raf-1 kinase activity in 1 to 5 min; Raf-1 kinase activity peaked after 5-10 min (FIG. 12A). The measurement of Raf-1 activity was based upon phosphorylation of MEK, thereby directly demonstrating the role of Raf-1 in initiation of the MAPK pathway in astrocytes. Increased Raf-1 enzyme activity was accompanied by dephosphorylation of Ser 259, an inhibitory phosphate site detected by a specific anti-Raf (Ser 259) antibody (FIG. 12B). The data demonstrate that RANTES stimulates Raf-1 activation in astrocytes.

To examine the effects of Raf-1 on induction of chemokine or cytokine transcripts, primary astrocytes were pre-treated with each of a series of increasing doses of Raf-1 inhibitor I prior to stimulation with RANTES.

After 3 h stimulation, RNA was prepared and examined for chemokine/cytokine transcription by RPA. Treatment with the Raf-1 inhibitor blocked gene expression in a dose dependent fashion (FIG. 12C). The Raf-1 inhibitor also blocked MEK and erk1/2 phosphorylation induced by RANTES linking Raf-1 to the MAPK pathway and to production of proinflammatory mediators in astrocytes. All concentrations of this inhibitor failed to affect astrocyte viability or expression of the housekeeping genes, L32 and GAPDH.

### Example 15

### Effects of Dominant Negative and Constitutively Active Raf

To examine the Raf dependence of RANTES-stimulated activation of the MIP-2 promoter, dominant negative and constitutively active mutants of Raf were examined. The phosphorylation site (Ser 621) required for kinase activity was mutated, resulting in a kinase defective protein (Mischak, et al. 1996. Mol Cell Biol 16(10), 5409-18; Morrison, et al., 1993. J Biol chem 268(23), 17309-16). Astrocytes were cotransfected with the luciferase-MIP-2 promoter construct along with wild type or mutant Raf. The cotransfected cells were stimulated with RANTES and monitored for luciferase reporter activity.

Dominant negative Raf was found to specifically suppress reporter activity (FIG. 12D) demonstrating the importance of this enzyme in regulating the transcription of the chemokine MIP-2. Constitutively active mutant Raf was sufficient to induce transcription form the MIP-2 promoter (FIG. 12D). The data demonstrate a key role for Raf in controlling RANTES-mediated astrocyte gene expression.

### Example 16

### Interaction Between PKA and MAPK Pathways

To determine whether there is an interrelationship between the cAMP/PKA and the Raf/MAPK pathways, astrocytes were treated with a series of increasing doses of each of the PKA inhibitors H-89, Rp-8-bromo-cAMP or PKI, and cells were harvested to monitor Raf-1 kinase activity.

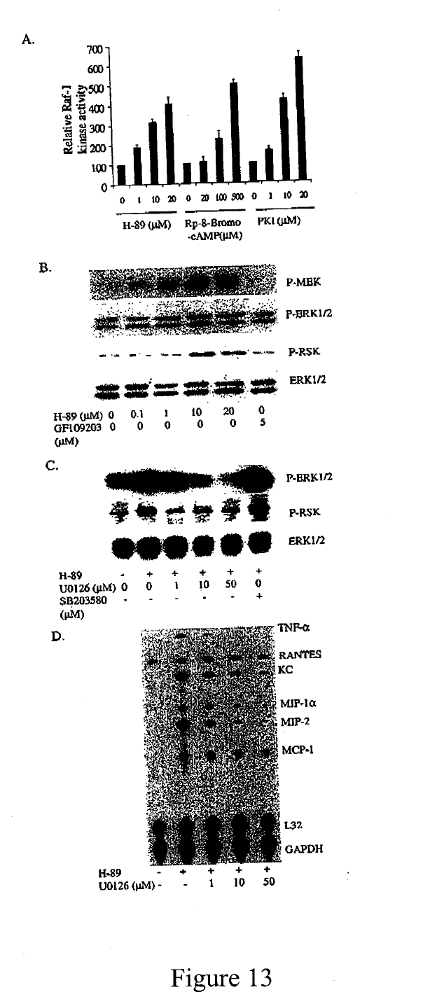

Inhibitors of PKA were found to increase Raf-1 kinase activity (FIG. 13A) in a dose dependent fashion, and to decrease phosphorylation of Raf-1 on Ser 259. These findings indicate that PKA acts upstream of Raf-1 in the RANTES signaling pathway.

H-89 treatment also induced MEK, erk1/2 and RSK phosphorylation in a dose dependent fashion (FIG. 13B). As a control GF109203, an inhibitor of protein kinase C, failed to stimulate MEK phosphorylation (FIG. 13B).

To examine the effects of the MAPK pathway on the induction of proinflammatory mediators, astrocytes were pretreated with increasing doses of MEK inhibitor U0126, prior to stimulation with RANTES or H-89.

Treatment with 10-50 μM U0126 blocked erk1/2 and RSK phosphorylation induced by H-89 (FIG. 13C). As a control, SB203580, an inhibitor of p38, was found to fail to block H-89 induced erk1/2 and RSK phosphorylation. U0126 also inhibited H-89 induced chemokine/cytokine transcription in a dose dependent manner (FIG. 13D). Occasional batches of astrocytes displayed high background levels of RANTES mRNA (FIG. 13D). Treatment with U0126 failed to diminish this background level of RANTES transcript, indicating that the effects of U0126 are activation specific. In addition, neither U0126 nor Raf-1 inhibitor decreased PKA activity. Therefore, PKA was shown to negatively regulate RANTES-induced gene transcription through inhibition of the Raf-1/MAPK pathway.

What is claimed is:

1. A method of reducing inflammatory responses in parenchymal cells of the central nervous system (CNS) of a subject, the method comprising providing the subject with an inhibitor of binding of a RANTES -related chemokine to a RANTES receptor in the CNS, such that RANTES signal transduction and amplification of chemokine gene expression are inhibited, thereby reducing inflammatory responses in the cells.

2. A method according to claim 1, wherein the chemokine is selected from the group consisting of RANTES, eotaxin, MIP-1α and MIP-1β.

3. A method according to claim 1, wherein the inhibitor is provided directly to the CNS.

4. A method according to claim 3, wherein the CNS is providing an additional agent that permeabilizes the blood-brain barrier.

5. A method according to claim 4, wherein delivering the inhibitor is providing the inhibitor in a CNS implant.

6. A method of obtaining an agent that inhibits up-regulation of expression of a proinflammatory gene in a population of astrocytes, the method comprising:
providing a sample of activated astrocytes with at least one candidate agent;
testing the candidate for ability to inhibit signal transduction of the RANTES/RSK pathway; and
identifying the candidate as an inhibitor of a step in the pathway of the sample of astrocytes in comparison with a control sample of astrocytes not provided with the candidate and otherwise identical, such that the candidate is an inhibitor of up-regulation of a proinflammatory gene in astrocytes.

7. A method according to claim 6, wherein the activated astrocytes are pretreated with a chemokine selected from the group consisting of RANTES, eotaxin, MIP-1α and MIP-1β.

8. A method according to claim 7, wherein the step of the pathway is RSK or PKA phosphorylation.

9. A method according to claim 8, wherein inhibiting the step is providing a mutant form of a RANTES-related chemokine.

10. A method according to claim 9, wherein the mutant form of the chemokine is a dominant negative mutant.

11. A method according to claim 8, wherein the inhibitor antagonizes chemokine binding to a CCR1 or CCR5 receptor.

12. A method according to claim 11, wherein the inhibitor antagonizes binding of HIV-1 to the receptor.

13. A method according to claim 11 wherein the inhibitor is selected from the group consisting of: APO-RANTES, sCH-C, and TAK-779.

14. A method of treating a subject having an inflammatory condition of the CNS, comprising providing an inhibitor obtained according to any of the methods of claims 7-13 of the RANTES/RSK signal transduction pathway; and administering a composition containing an effective dose of the inhibitor in a pharmaceutically acceptable excipient.

15. A method according to claim 14, wherein the inflammatory condition of the CNS is a demyelinating condition.

16. A method according to claim 15, wherein the demyelinating condition is multiple sclerosis or experimental allergic encephalomyelitis (EAE).

17. A method according to claim 15, wherein the demyelinating condition is selected from the group consisting of a post-vaccination condition, post-viral infection condition, and a post-anti TNF treatment condition.

18. A method according to claim 14, wherein the inflammatory condition of the CNS is a neurodegenerative disease.

19. A method according to claim 18, wherein the neurodegenerative disease is Alzheimer's disease or Parkinson's disease.

20. A method according to claim 14, wherein the inflammatory condition of the CNS is selected from meningitis, cerebritis, brain and spinal cord injury, and stroke.

21. A method according to claim 16, wherein the composition further comprises an additional therapeutic agent.

22. A method according to claim 21, wherein the composition further comprises β-interferon.

23. A method according to claim 21, wherein the composition further comprises a random linear amino acid copolymer.

24. A method according to claim 21, wherein the composition further comprises Copaxone®.

25. A method of screening a library comprising a plurality of compounds to identify an inhibitor of RANTES/RSK signal transduction in a parenchymal cell of the CNS, the method comprising:
providing a cell with a RANTES-related chemokine and at least one of the compounds; and
analyzing the cell for expression of a gene that is up-regulated in response to chemokine treatment, wherein decreased expression of the gene in the presence of the compound, compared to that in a control cell similarly treated with chemokine but in the absence of the compound, indicates that the compound is an inhibitor of the pathway.

26. A method according to claim 25, wherein the RANTES-related chemokine is selected from the group consisting of RANTES, eotaxin, MIP-1α and MIP-1β.

27. A method according to claim 25, wherein the parenchymal cell is an astrocyte.

28. A method according to claim 25, wherein the gene that is up-regulated encodes an adhesion molecule.

29. A method according to claim 28, wherein the adhesion molecule is selected from the group consisting of ICAM-1, CX3CR1, and CXCR4.

30. A method according to claim 25, wherein analyzing the cell for expression of the gene further includes measuring an RNA transcript of the gene.

31. A method according to claim 25, wherein analyzing the cell for expression of the gene is measuring a protein product of the gene.

32. A method according to claim 31, wherein measuring the protein product is further measuring the protein antigenically.

33. A method according to claim 31, wherein measuring the protein product is measuring the protein functionally.

34. A method according to claim 32, wherein measuring the protein antigenically is performing a western blot.

35. A method according to claim 33, wherein measuring the protein functionally is measuring a marker enzyme

36. A method according to claim 35, wherein the marker enzyme is encoded by a fusion of the gene and a nucleic acid encoding the marker enzyme.

37. A method according to claim 35, wherein the marker enzyme is selected from the group consisting of luciferase and β-galactosidase.

38. A method according to claim 33, wherein measuring the protein functionally is assaying for expression of a fusion of the gene with a non-enzymatic marker protein.

39. A method according to claim 38, wherein the non-enzymatic marker protein is a colored fluorescent protein.

40. A method according to any of claims 25-39, wherein the gene encodes a protein which is selected from the group consisting of: TNF-α, RANTES, KC, IL-6, MIP-1α, MIP-2, MCP-1, ICAM-1, CX3CR1, and CXCR4.

41. A method of screening a plurality of compounds to identify an inhibitor of the RANTES/RSK pathway in parenchymal cells of the CNS, the method comprising:
providing a RANTES-related chemokine and at least one compound of the plurality to a sample of the cells; and
analyzing the sample of cells for phosphorylation of a protein of the pathway, wherein a change in phosphorylation of the protein in the presence of the compound, compared to that in a control sample of the cells similarly treated with the chemokine in the absence of the compound indicates that the compound is an inhibitor of the pathway.

42. A method according to claim 41, wherein the protein is selected from the group of RSK, Raf-1, MEK, and PKA.

43. A method of screening a library of compounds to identify a candidate compound that is an inhibitor of the RANTES/RSK pathway in parenchymal cells of the CNS, the method comprising:
providing a first cell extract from a sample of the parenchymal cells that have been pre-treated with a RANTES-related chemokine, and a second cell extract from otherwise identical control parenchymal cells which have not been pre-treated with the chemokine;
providing at least one candidate compound to the first and second extracts; and
assaying the first and second extracts for activity of a protein in the RANTES/RSK/Raf-1/PKA pathway in the presence and absence of the candidate inhibitor, wherein decreased function of the protein in the first cell extract in the presence of the compound, compared to that of the first cell extract in the absence of the compound and the second cell extract, indicates that the compound is an inhibitor of the pathway.

44. A method according to claim 43, wherein the activity of the protein is a kinase.

45. A method according to claim 43, wherein cells are pretreated with chemokine at a concentration of about 1 nm.

46. A method according to claim 43, wherein cells are pretreated with chemokine at a concentration of about 2 nm.

47. A method according to claim 43, wherein cells are pretreated with chemokine at a concentration of about 10 nm.

48. A method according to claim 43, wherein cells are pretreated with chemokine at a concentration about 100 ng/ml.

49. A method according to any of claims 45-48, wherein pretreated cells are pre-treated with chemokine for at least 5 minutes.


SEQUENCE LISTING (5 sequences)

SEQ ID NO: 1 - DNA, 28 bp, Artificial Sequence
  Description of Artificial Sequence PCR Primer Sequence
gcgaattcaccatgatcccagccacccg

SEQ ID NO: 2 - DNA, 29 bp, Artificial Sequence
  Description of Artificial Sequence PCR Primer Sequence
gctctagattacttggggacaccttttag

SEQ ID NO: 3 - DNA, 21 bp, Artificial Sequence
  Description of Artificial Sequence PCR Primer Sequence
atccgagggaaaggcttcgac

SEQ ID NO: 4 - DNA, 21 bp, Artificial Sequence
  Description of Artificial Sequence PCR Primer Sequence
gagcagaggaaggctcattgg

SEQ ID NO: 5 - protein, 7 aa, Artificial Sequence
  Description of Artificial Sequence Biotinylated Kemptide
LRRASLG